(19) United States
(12) Patent Application Publication
(10) Pub. No.: US 2023/0317235 A1
(43) Pub. Date: Oct. 5, 2023

(54) EFFECTIVE MOBILITY

(71) Applicant: INTERNATIONAL BUSINESS MACHINES CORPORATION, ARMONK, NY (US)

(72) Inventor: Pritish Ranjan Parida, Cortlandt Manor, NY (US)

(21) Appl. No.: 17/656,984

(22) Filed: Mar. 29, 2022

(51) Int. Cl. G16H 20/30

(52) U.S. Cl. CPC G16H 20/30

(57) ABSTRACT

A method, computer system, and a computer program product for effective mobility scoring is provided. The present invention may include receiving an acceleration data associated with a user. The present invention may also include determining a respective activity state of a plurality of activity states for the user based on the received acceleration data. The present invention may also include recording an amount of time spent in a respective activity state of a plurality of activity states. The present invention may further include determining an effective mobility score of the user for a specified time-window based on calculating a weighted sum of time spent in the plurality of activity states.

### BACKGROUND

The present invention relates generally to the field of computing, and more particularly to user mobility analysis.

The measurement and treatment of pain is one of the most challenging healthcare issues. Patients suffering from chronic pain experience significant changes in different dimensions of their life, especially in the aspects of their overall mobility and self-care. Mobility may refer to one's ability to move freely and easily. Physiologically, mobility is a manifestation of a functional integration of skeletal, muscular, nervous, circulatory, and respiratory systems. Thus, mobility may represent critical clinical evidence in assessing physical and cognitive health, for example, progression of neuro-degenerative diseases, quality of life, risk of fall, ability of independent living, and frailty of pre- and post-surgery patients.

### SUMMARY

Embodiments of the present invention disclose a method, computer system, and a computer program product for effective mobility scoring. The present invention may include receiving an acceleration data associated with a user. The present invention may also include determining a respective activity state of a plurality of activity states for the user based on the received acceleration data. The present invention may also include recording an amount of time spent in a respective activity state of a plurality of activity states. The present invention may further include determining an effective mobility score of the user for a specified time-window based on calculating a weighted sum of time spent in the plurality of activity states.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is a schematic block diagram of a mobility assessment environment according to at least one embodiment;

FIG. 3 is an operational flowchart illustrating a process for mobility assessment according to at least one embodiment;

FIG. 4 is a block diagram of internal and external components of computers and servers depicted in FIG. 1 according to at least one embodiment;

FIG. 5 is a block diagram of an illustrative cloud computing environment including the computer system depicted in FIG. 1, in accordance with an embodiment of the present disclosure; and

FIG. 6 is a block diagram of functional layers of the illustrative cloud computing environment of FIG. 5, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, Python, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for determining the effective mobility of a user. As such, the present embodiment has the capacity to improve the technical field of pain management by providing an objective and passive measurement of pain. More specifically, a mobility assessment program may receive an acceleration data associated with a user. Then, the mobility assessment program may calculate an activity intensity of the user based on the received acceleration data. Next, the mobility assessment program may record an amount of time spent in a respective activity state of a plurality of activity states. In one embodiment, the mobility assessment program may identify the respective activity state based on the calculated activity intensity. Then, the mobility assessment program may determine an effective mobility score of the user for a specified time-window based on calculating a weighted sum of time spent in the plurality of activity states. Thereafter, the mobility assessment program may automatically quantify a pain intensity experienced by the user in the specified time-window based on the determined effective mobility score of the user.

As described previously, the measurement and treatment of pain is one of the most challenging healthcare issues. Patients suffering from chronic pain experience significant changes in different dimensions of their life, especially in the aspects of their overall mobility and self-care. Mobility may refer to one's ability to move freely and easily. Physiologically, mobility is a manifestation of a functional integration of skeletal, muscular, nervous, circulatory, and respiratory systems. Thus, mobility may represent critical clinical evidence in assessing physical and cognitive health, for example, progression of neuro-degenerative diseases, quality of life, risk of fall, ability of independent living, and frailty of pre- and post-surgery patients.

Traditionally, pain is clinically measured using a process where patients are asked to rate their pain intensity on a scale between 0 to 10 using a Numeric Rating Scale (NRS) or using a Visual Analog Scale (VRS). However, this traditional self-reporting process for measuring pain is highly subjective. For example, two people may have different pain thresholds and what a first person perceives as high levels of pain might not be perceived the same by a second person.

Traditional pain measurement techniques using self-reporting may also be inaccurate. For example, self-reporting may fail to capture the dynamics of day-to-day pain intensity, which is important in understanding disease progression and therapeutic response. Self-reporting may also lead to downplaying the pain intensity or the chronic pain symptoms.

Therefore, it may be advantageous to, among other things, provide a way to passively and objectively measure a user's chronic pain intensity using the user's movement data captured using a wearable sensor.

Accordingly, the forthcoming detailed description presents a system for assessing user mobility and pain intensity using only a user movement (e.g., acceleration) data gathered by, for example, a wearable device. Benefits of the proposed motion tracking system include, to name a few, low cost, scalability, and the ability to increase data collection type and rate. Moreover, benefits of the aggregation and analysis architecture further include support for a wide range of assessments, such as, for example, progression of neuro-degenerative disease, strength and endurance, cardiovascular fitness, quality-of-life, fall risk, and frailty, on a regular basis (e.g., daily, hourly, daily, weekly), as well as the ability to create predictive models through machine learning of the data.

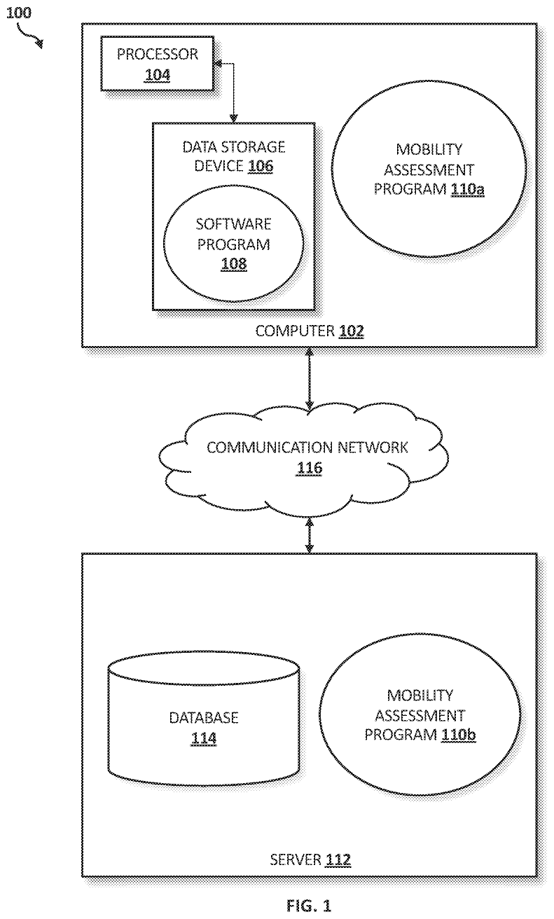

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a mobility assessment program 110a. The networked computer environment 100 may also include a server 112 that is enabled to run a mobility assessment program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

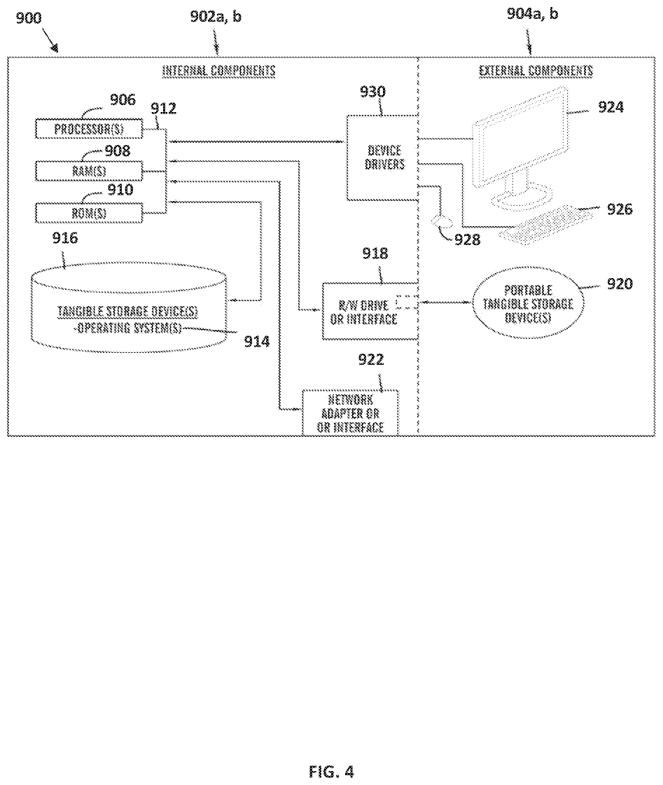

The client computer 102 may communicate with the server computer 112 via the communications network 116. The communications network 116 may include connections, such as wire, wireless communication links, or fiber optic cables. As will be discussed with reference to FIG. 4, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the mobility assessment program 110a, 110b may interact with a database 114 that may be embedded in various storage devices, such as, but not limited to a computer/mobile device 102, a networked server 112, or a cloud storage service.

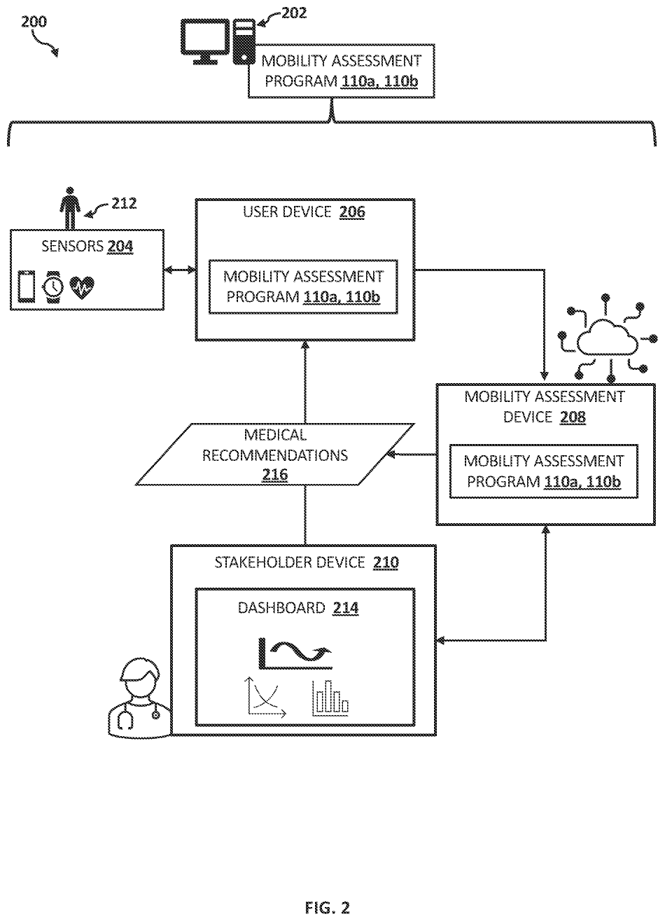

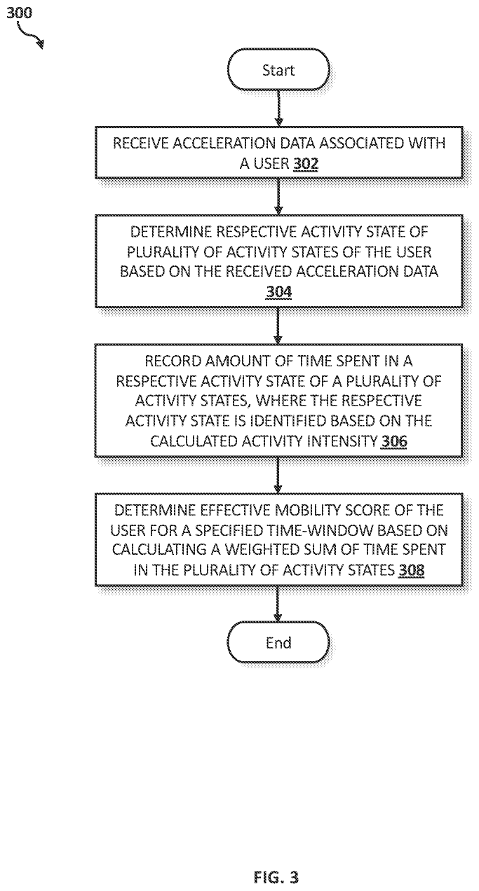

According to the present embodiment, a user using a client computer 102 or a server computer 112 may use the mobility assessment program 110a, 110b (respectively) to determine an effective mobility score of a user for a specified time-window based on calculating a weighted sum of time spent in a plurality of activity states based on acceleration data. The disclosed embodiments are explained in more detail below with respect to FIGS. 2 and 3.

Referring now to FIG. 2, a schematic block diagram of a mobility assessment environment 200 implementing the anomaly detection program 110a, 110b according to at least one embodiment is depicted.

According to one embodiment, the mobility assessment environment 200 may include one or more components (e.g., client computer 102; server computer 112; communication network 116) of the computer environment 100 discussed above with reference to FIG. 1. While programming and data of the exemplary embodiments may be stored and accessed remotely across several servers via the communication network 116, programming and data of the exemplary embodiments may alternatively or additionally be stored locally on as few as one physical computing device or amongst other computing devices than those depicted.

According to one embodiment, the mobility assessment environment 200 may include a computer system 202 having a tangible storage device and a processor that is enabled to run the mobility assessment program 110a, 110b. In one embodiment, the computer system 202 may include one or more sensors 204, one or more user devices 206 (e.g., client computer 102), one or more mobility assessment devices 208 (e.g., server computer 112), and one or more stakeholder devices 210 (e.g., client computer 102), which may all be interconnected via communication network 116. In various embodiments, the user device 206, the mobility assessment device 208, and/or the stakeholder device 210 of the computer system 302 may include a workstation, a personal computing device, a laptop computer, a desktop computer, a computing server, a thin-client terminal, a tablet computer, a smartphone, a smart watch or other smart wearable device, or any other electronic device or computing system capable of sending and receiving data to and from other computing devices. In at least one embodiment, the mobility assessment device 208 may also operate in a cloud computing service model, such as Software as a Service (SaaS), Platform as a Service (PaaS), or Infrastructure as a Service (IaaS). In one embodiment, the mobility assessment device 208 may also be located in a cloud computing deployment model, such as a private cloud, community cloud, public cloud, or hybrid cloud.

In one embodiment, the mobility assessment program 110a, 110b may include a single computer program or multiple program modules or sets of instructions being executed by the processor of the computer system 202. The mobility assessment program 110a, 110b may include routines, objects, components, units, logic, data structures, and actions that may perform particular tasks or implement particular abstract data types. The mobility assessment program 110a, 110b may be practiced in distributed cloud computing environments where tasks may be performed by local and/or remote processing devices which may be linked through the communication network 116. In one embodiment, the mobility assessment program 110a, 110b may include program instructions that may be collectively stored on one or more computer-readable storage media. As such, in various embodiments, a first instance of the mobility assessment program 110a, 110b may be implemented in the user device 206 and a second instance of the mobility assessment program 110a, 110b may be implemented in the mobility assessment device 208. In at least one embodiment, although not specifically shown in the figures, a third instance of the mobility assessment program 110a, 110b may also be implemented in the stakeholder device 210.

In exemplary embodiments, the sensors 204 may include one or more devices, e.g., wearable devices/sensors, capable of collecting data. In particular, the sensors 204 may be configured to collect data that may be analyzed to estimate a motion and mobility of a user 212, including acceleration, heartrate, location, center of mass, body frame, user state, body orientation, skeleton, joints, ECG (electrocardiogram) signal, EMG (electromyography) signal, PPG (Photoplethysmography) signal, and/or Blood oxygen saturation.

In one embodiment, the sensors 204 may be a wearable smart device (e.g., a smart watch), adhesive patch, belt, and/or smart clothing, that includes an accelerometer, heartrate monitor, gyroscope, electrodes (e.g., electrocardiogram/electromyography/electrodes), LED (e.g., photoplethysmography LEDS), and/or a Global Positioning System (GPS). In embodiments, the sensors 204 may communicate with the communication network 116 or with the user device 206 through means such as WiFi, Bluetooth® (Bluetooth and all Bluetooth-based trademarks and logos are trademarks or registered trademarks of Bluetooth SIG, Inc. and/or its affiliates), and/or Near Field Communication (NFC). In various embodiments, the sensors 204 may communicate directly with the mobility assessment device 208 via communication network 116.

According to one embodiment, the sensors 204 may be integrated into the user device 206 (e.g., smart watch) such that the user device 206 may be capable of collecting data that may be analyzed to estimate a motion and mobility of a user 212, including acceleration, heartrate, location, center of mass, body frame, user state, body orientation, skeleton, joints, ECG (electrocardiogram) signal, EMG (electromyography) signal, PPG (Photoplethysmography) signal, and/or Blood oxygen saturation.

According to one embodiment the user device 206 and/or the sensors 204 may be associated with the user 212. In one embodiment, the user 212 may include a patient with chronic pain or another disease which may require tracking the patient's mobility and pain to generate treatment recommendations. In some embodiments, the user 212 may include anyone that wants to and/or needs to analyze one or more biomarkers, such as, for example, effective mobility.

In exemplary embodiments, the mobility assessment program 110a, 110b may be initially configured by receiving user consent from the user 212 to collect data as well as registration information based on, for example, log in credentials, internet protocol (IP) address, and/or media access control (MAC) address, via the user device 206 and the communication network 116. With respect to receiving user consent, the mobility assessment program 110a, 110b may allow the user 212 to manage the data collected and the manner in which the data may be collected, used, transferred, and/or distributed, as well as an option to opt out of such data collection. In any managing of user data, the mobility assessment program 110a, 110b may be configured to adhere to at least all applicable data handling and privacy protocols.

In addition to receiving user consent, the mobility assessor 132 may further receive user registration information, including demographic information, such as user name, date of birth, and/or location, as well as health and mobility related data. The health and mobility related data may be received via user/physician input, and/or reference to an electronic health/medical record, and may include one or more user health conditions and/or baseline user metrics. In one embodiment, if the sensors 204 are not integrated into the user device 206, configuration may also include establishing communication with the sensors 204 via, e.g., WiFi, Bluetooth® (Bluetooth and all Bluetooth—based trademarks and logos are trademarks or registered trademarks of Bluetooth SIG, Inc. and/or its affiliates), and/or NFC.

According to one embodiment, the mobility assessment program 110a, 110b may be implemented to receive user movement data from the sensors 204 (e.g., following user consent to having movement data collected). In one embodiment, the movement data may include acceleration data, herein defined as a rate of change in velocity over time, in any suitable rate format. In one embodiment, the sensors 204 may include a triaxial accelerometer implemented to simultaneously measure user acceleration in one or more orthogonal directions. For example, the triaxial accelerometer may be implemented to simultaneously measure user acceleration in three orthogonal directions (e.g., x-axis; y-axis; and z-axis).

According to one embodiment, the mobility assessment program 110a, 110b running on the mobility assessment device 208 may be implemented to receive the acceleration data (e.g., time series of raw triaxial acceleration data) directly from the sensors 204 via communication network 116. In another embodiment, the acceleration data may be first received by the mobility assessment program 110a, 110b running on the user device 206 from the sensors 204 (e.g., via NFC) before transmission to the mobility assessment device 208.

According to one embodiment, the acceleration data may be used to determine an activity state of the user 212 which may then be used to determine an effective mobility metric associated with the user 212. In one embodiment, the effective mobility metric may capture an overall strength or amplitude of user motion and may be determined by performing a spectrum analysis (e.g., frequency-domain analysis) of the received acceleration data. In this approach, the spectrum analysis produces a spectral power density as a function of frequency over a range of frequencies observed in the signal. For example, if the acceleration data is measured at 50 Hz (that is, 50 data samples every second), then the frequencies observed may range from 0 to 25 Hz (e.g., 0.5*50 Hz). For 30 Hz acceleration data sampling the range would be 0 to 15 Hz. The spectrum analysis may be performed at a regular time interval. For example, the acceleration data may be measured at 50 Hz and the spectrum analysis may be performed every 15 seconds on the acceleration data collected over the past 30 seconds. Next, the peak frequency (that is, frequency corresponding to the peak value of the spectral power density) may be identified. Depending upon the identified peak frequency an activity state may be assigned, as shown in Table 1, below.

TABLE 1

| | PEAK FREQUENCY |
|---|---|
| ACTIVITY STATE | THRESHOLDS |
| Rest | f ≤ 0.4 Hz |
| Low/Sedentary (Sedentary) | 0.4 Hz < f ≤ 0.8 Hz |
| Low-Medium/Mild (Mild) | 0.8 Hz < f ≤ 2 Hz |
| Medium/Moderate (Moderate) | 2 Hz < f ≤ 5 |
| Medium-High/Impact (Impact) | 5 < f ≤ 10 Hz |
| High/Jitter (Jitter) | f > 10 Hz |

According to one embodiment, the mobility assessment program 110a, 110b may identify the activity state of the user based on comparing the identified peak frequency with the peak frequency thresholds defining a set of activity states. Each corresponding bin for a respective activity state may include a minimum peak frequency threshold and a maximum peak frequency threshold. In one embodiment, the mobility assessment program 110a, 110b may identify the activity state by identifying the corresponding bin where the peak frequency associated with the user is greater than the minimum peak frequency threshold and less than or equal to the maximum peak frequency threshold.

According to one embodiment, the mobility assessment program 110a, 110b may additionally, or alternatively, use time-domain analysis to determine the activity state of the user 212 which may then be used to determine the effective mobility metric associated with the user 212. In this approach, the mobility assessment program 110a, 110b may first use the acceleration data to calculate an activity intensity (AI) of the user 212. In one embodiment, the AI may be calculated using the following Equation 1:

$$A I = 100 ⋆ \sqrt{\frac{( {σ}_{ax}^{2} + {σ}_{ay}^{2} + {σ}_{az}^{2}}{3}} \qquad Eq . 1$$

According to one embodiment, ax may be referred to as acceleration measured in the x-axis, ay may be referred to as acceleration measured in the y-axis, and az may be referred to as acceleration measured in the z-axis, where the acceleration measurements may be simultaneous measurements in three orthogonal directions. In one embodiment, the acceleration data used to measure the AI may be sampled at regular time intervals. For example, the acceleration data may be sampled 30 times every second (that is, sampled at 30 Hz) and the AI may be computed every 15 seconds, using 30 seconds processing window. In other embodiments, the acceleration data may be sampled using any suitable regular time interval.

According to one embodiment, the AI calculation may be implemented by the mobility assessment program 110a, 110b as a data reduction strategy to transform the large amount of raw acceleration data collected by the sensors 204 into the various activity states of the user 212 over a specified period of time (e.g., 24 hours). In one embodiment, the raw acceleration data may be transferred (e.g., via communication network 116) from the sensors 204 and/or the user device 206 to the mobility assessment device 208 and the mobility assessment program 110a, 110b running on the mobility assessment device 208 may calculate the AI. In another embodiment, the mobility assessment program 110a, 110b may implement edge computing techniques to process (e.g., calculate the AI) the raw acceleration data locally (e.g., via user device 206) to reduce the amount of data that is transferred to the mobility assessment device 208.

According to one embodiment, the mobility assessment program 110a, 110b may define a set of activity states (Table 2, below) which may be based on certain thresholds observed during mobility assessment tests, e.g., six minute walk test (6MWT), for the user 212 or across several patients (e.g., having same or similar disease conditions as the user 212) over multiple clinical visits. In one embodiment, the AI thresholds may be implemented as wide enough to capture variability across patients and/or be implemented as patient (e.g., user 212) specific as well.

TABLE 2

| | ACTIVITY INTENSITY |
|---|---|
| ACTIVITY STATE | THRESHOLDS |
| Rest | AI ≤ 10 |
| Low/Sedentary (Sedentary) | 10 < AI ≤ 50 |
| Low-Medium/Mild (Mild) | 50 < AI ≤ 150 |
| Medium/Moderate (Moderate) | 150 < AI ≤ 250 |
| Medium-High/Impact (Impact) | 250 < AI ≤ 400 |
| High/Jitter (Jitter) | AI > 400 |

According to one embodiment, the AI thresholds may define the bins for respective activity states. In one embodiment, the AI thresholds may be determined from data (e.g., acceleration data) collected from a similar device (e.g., smart watch) during a fitness assessment test performed by the user 212 and/or a patient with the same or similar condition (e.g., disease; health history) as the user 212. For example, the AI thresholds may be a first set of thresholds for the user 212 having chronic pain and may be a second set of different thresholds for the user 212 recovering from a knee surgery. According to one embodiment, the AI thresholds may be different for different devices (e.g., sensors 204). For example, the AI thresholds may be different for a body worn-patch and a wrist-worn smart watch.

According to one embodiment, the mobility assessment program 110a, 110b may define six activity states as shown in Table 2. In other embodiments, the mobility assessment program 110a, 110b may define any suitable number of activity states.

According to one embodiment, the mobility assessment program 110a, 110b may compute (e.g., via user device 206 or mobility assessment device 208) an amount of time (e.g., fraction of time) spent in minutes in a given activity state within the last hour based on the calculated AI. In other embodiments, the mobility assessment program 110a, 110b may compute (e.g., via user device 206 or mobility assessment device 208) the amount of time (e.g., fraction of time) spent in minutes or hours in a given activity state within the last day (e.g., 24 hours) based on the calculated AI. In one embodiment, the mobility assessment program 110a, 110b may record (e.g., in data storage device 106 and/or in database 114) the amount of time spent in a respective activity state of the various activity states, where the respective activity state may be identified based on the calculated AI.

According to one embodiment, the mobility assessment program 110a, 110b may then compute (e.g., via user device 206 or mobility assessment device 208) the effective mobility of the user 212. In one embodiment, the effective mobility may refer to a weighted sum of the fraction of time spent in different tiers of activity (e.g., the activity states). The effective mobility (EM) may be expressed as Equation 2, below:

$$E M = \frac{{\sum }_{k = 0}^{N} ( {C}_{k} * {t}_{k} )}{{\sum }_{k = 0}^{N} {C}_{k}} \qquad Eq . 2$$

According to one embodiment, the effective mobility may be dependent on the definition of each of the activity states N and the associated AI thresholds. Thus, in one embodiment, the effective mobility may be a patient-specific (e.g., user 212) metric if the AI thresholds are determined from data (e.g., acceleration data) collected from a fitness assessment test performed by the user 212 and/or a patient with the same or similar condition (e.g., disease; health history) as the user 212.

According to one embodiment, in Equation 2 above, tₖ may refer to the fraction of time spent in the k-th activity state and Cₖ may refer to the associated weighting factor as shown in Table 3, below:

TABLE 3

| ACTIVITY | | |
|---|---|---|
| STATE | Cₖ | EXAMPLES |
| Rest | 0 | Resting State, little movement |
| Sedentary | 1 | Using a mobile phone, remote control, or typing |
| Mild | 2 | Dressing, moving around, slow walking, stretching, |
| | | and walking in a pool |
| Moderate | 3 | Brisk walking or 6-minute walk test |
| Impact | 4 | Running, swimming, exercising |
| Jitter | 5 | Sustained high frequency movements. Typically, |
| | | hand movements, e.g., cooking, cleaning, hand |
| | | tremors, seizures |

According to one embodiment, the fraction of time (tₖ) may be computed as a fraction of an hour or a fraction of a day as discussed previously. For example, if the effective mobility is calculated daily (e.g., once per day), the time in each activity state may be calculated and recorded as the fraction of a day. According to one embodiment, the effective mobility metric may be normalized by the device (e.g., sensors 204) worn duration to improve the signal strength.

According to one embodiment, the mobility assessment program 110a, 110b may calculate an hourly index of effective mobility for the user 212, where the fraction of time (tₖ) may be computed as a fraction of an hour (e.g., minutes per hour). In embodiments, the hourly index of effective mobility may indicate an objective level of user mobility per hour, and may be determined based on breaking down the effective mobility calculation into a per hour determination to normalize the effective mobility at the hour level, as expressed by Equation 3, below:

$$E M = \frac{\frac{{\sum }_{k = 0}^{N} ( {C}_{k} * {t}_{k} )}{{\sum }_{k = 0}^{N} {C}_{k}}}{60 minutes} \qquad Eq . 3$$

The mobility assessment program 110a, 110b may use a time series of the acceleration data in order to isolate different hours of the day and discern an hourly index of effective mobility. The hourly index of mobility may indicate variability in times at which the user 212 is mobile.

Additionally, or alternatively, the mobility assessment program 110a, 110b may also calculate a daily index of effective mobility for the user 212, where the fraction of time (tₖ) may be computed as a fraction of a day (e.g., minutes per day; hours per day). In embodiments, the daily index of effective mobility may indicate an objective level of user mobility per day, and may be determined based on breaking down the effective mobility calculation into a per day (e.g., 24 hours) determination to normalize the effective mobility at the day (e.g., 24 hours) level, as expressed by Equation 4, below:

$$E M = \frac{\frac{\frac{{\sum }_{k = 0}^{N} ( {C}_{k} * {t}_{k} )}{{\sum }_{k = 0}^{N} {C}_{k}}}{60 minutes}}{24 hours} \qquad Eq . 4$$

According to one embodiment, the mobility assessment program 110a, 110b may determine the functional relationship between pain intensity and effective mobility for the user 212. In one embodiment, the relationship between effective mobility and pain intensity for a given user 212 may be expressed as a monotonic function where the pain intensity only increases with an increase in effective mobility, or the pain intensity only decreases with an increase in effective mobility. In one embodiment, the monotonic function expressing the relationship between the pain intensity (P) and the effective mobility (EM) may be represented by Equation 5, below:

P=a*EM+b (5)

According to one embodiment, in Equation 5 above, the values for P may range from 0 to 10 and the values for EM may range from 0 to 1. In at least one embodiment, the values of P and EM may include any other suitable ranges. According to one embodiment, in Equation 5 above, a and b may include constants. In one embodiment, a and b may include constants that are patient-specific (e.g., specific to user 212). Constant a may indicate the slope of the functional relationship between pain intensity and effective mobility. In one embodiment, constant a may represent the rate at which pain intensity changes with a change in effective mobility. In one embodiment, a negative value for a may indicate that pain decreases with increase in effective mobility and vice versa. Constant b may refer to the reference pain intensity. In one embodiment, if the slope (e.g., constant a) is negative, then b may have a value closer to max pain intensity (e.g., closer to 10) and if slope (a) is positive, then b may have a value closer to min pain intensity (e.g., closer to 0). For example, for patient A, a=3 and b=0.5 and for patient B, a=−4 and b=9.

Alternately, the relationship between effective mobility and pain intensity for a given user 212 may be expressed as a non-monotonic where the pain intensity decreases with an increase in effective mobility up to a certain threshold value of effective mobility and beyond that, the pain intensity increases with an increase in effective mobility or vice versa (e.g., pain intensity increases with an increase in effective mobility up to a certain threshold value of effective mobility and beyond that, the pain intensity decreases with an increase in effective mobility). In one embodiment, the non-monotonic function expressing the relationship between the pain intensity (P) and the effective mobility (EM) may be represented as a piece-wise function shown in Equation 6, below:

$$P = \{ \begin{matrix}{a}_{1} * E M + {b}_{1} & for E M \leq {a}_{0} \\ {b}_{2} + \frac{c}{1 + {e}^{- {a}_{2} * ( E M - {a}_{0} )}} & for E M > {a}_{0}\end{matrix} \qquad Eq . 6$$

According to one embodiment, in Equation 6 above, a₀, a₁, a₂, b₁, b₂, and c may include constants. In one embodiment, a₀, a₁, a₂, b₁, b₂, and c may include constants that are patient-specific (e.g., specific to user 212).

Aspects of the present disclosure relates to implementing the mobility assessment program 110a, 110b to automatically determine whether the relationship between effective mobility and pain is monotonic or non-monotonic for the given user 212 using a machine learning model.

According to one embodiment, the mobility assessment program 110a, 110b may provide at least two methods for determining whether the relationship between effective mobility and pain is monotonic or non-monotonic for the given user 212.

In one embodiment, the mobility assessment program 110a, 110b may determine whether the relationship between effective mobility and pain is monotonic or non-monotonic for the given user 212 based on training or identifying a machine learning model that correlates the effective mobility metric and pain intensity level. In particular, the mobility assessment program 110a, 110b may first train the machine learning model by defining a training period (e.g., first 15 days) and prompting the user 212 to report their pain intensity levels (e.g., between 0 to 10) on a regular basis (e.g., once per day or twice per day or once every other day) while acceleration is automatically collected using sensors 204. In general, for real world application all the historical data for pain intensity and corresponding effective mobility data may be used to train the model/identify the functional relationship for a given patient.

Once trained, the mobility assessment program 110a, 110b may implement the machine learning model and the effective mobility data (e.g., current EM) to predict (e.g., quantify) disease severity and/or pain intensity level for the user 212 without requiring the user 212 to report their subjective assessments of disease severity and/or pain intensity level.

In another embodiment, if there is no historical data for the given user 212 (e.g., historical pain intensity and corresponding effective mobility data), the mobility assessment program 110a, 110b may identify one or more cohorts of the user 212 where the cohort data includes a relationship between effective mobility and pain intensity. In one embodiment, a cohort may be defined based on age, gender, ethnicity, comorbidity, and/or disease condition being similar to that of the user 212. The known relationship between effective mobility and pain intensity available for the most similar cohort may be used for the user 212 until a sufficient amount (e.g., 20 data points) of pain intensity reports and corresponding effective mobility data is collected for the user 212. Thereafter, the mobility assessment program 110a, 110b may be implemented to use the user 212 data to determine the user-specific relationship between the effective mobility and the pain intensity.

According to one embodiment, the mobility assessment program 110a, 110b may generate and transmit a dashboard 214 to one or more stakeholder devices 210. According to one embodiment, the dashboard 214 may include a summary of the progress made by the user 212. For example, the dashboard 214 may depict one or more visualizations (e.g., line graphs, pie charts, calendars) of effective mobility results in various time periods (e.g., hourly, daily, weekly, monthly). According to one embodiment, by providing the dashboard 214 to one or more stakeholder devices 210, the mobility assessment program 110a, 110b may enable remote monitoring of the health progression of the user 212 by subject matter experts (SMEs), such as, for example, the user's physician or medical team. In various embodiments, the mobility assessment program 110a, 110b may also provide access to the dashboard 214 to other stakeholders such as, for example, the insurance provider for the user 212. In at least one embodiment, the mobility assessment program 110a, 110b may also provide the user 212 with access to the dashboard 214 via the user device 206.

In one embodiment, the predicted pain intensity level and/or the predicted disease severity level may include automatically quantifying the pain intensity level and/or the disease severity level experienced by the user in the specified time-window (e.g., pain intensity level each hour or each day) based on the effective mobility score of the user 212. According to one embodiment, the mobility assessment program 110a, 110b may generate one or more medical recommendations 216 based on the predicted (e.g., automatically quantified) pain intensity level and/or the predicted disease severity experienced by the user 212 and the functional relationship between the effective mobility score of the user 212 and the automatically quantified pain intensity experienced by the user 212.

According to one embodiment, the mobility assessment program 110a, 110b may generate therapy recommendations (e.g., medical recommendations 216) based on latest/current pain intensity or disease severity level (e.g., determined based on effective mobility score) and inputs from SMEs, such as, the physician of the user 212. In at least one embodiment, the medical recommendations 216 may include therapeutic interventions needed based on predicted pain intensity and/or disease severity level using current effective mobility data and learnt patient-specific functional relationship between effective mobility and pain intensity and/or disease severity level.

According to one embodiment, the mobility assessment program 110a, 110b may use the functional relationship between pain intensity and effective mobility for a given patient (e.g., user 212) to provide an activity or mobility recommendation (e.g., medical recommendations 216) specific to the patient. For example, if for a given patient the relationship between pain intensity and effective mobility is such that the pain increases with increase in effective mobility, the mobility assessment program 110a, 110b may generate the medical recommendations 216 to indicate a reduction in any strenuous activities or to increase the resting time between subsequent activities.

In another scenario, if for a given patient the relationship between pain intensity and effective mobility is such that the pain decreases with increase in mobility, then the mobility assessment program 110a, 110b may generate the medical recommendations 216 to indicate walking for additional X minutes each day (e.g., where X may indicate 15, 30, 60 minutes) or to perform the user's favorite activity for additional X minutes each day or to exercise a X minutes longer each day or to be otherwise X minutes more active each day.

In another scenario, if for a given patient (e.g., user 212) the relationship between pain intensity and effective mobility is non-monotonic such that the pain intensity increases with effective mobility beyond a certain pain or effective mobility threshold, then based on the current pain level for the patient, the mobility assessment program 110a, 110b may generate the medical recommendations 216 to indicate either increasing or decreasing the patient's effective mobility.

The mobility assessment program 110a, 110b may then transmit the medical recommendations 216 to the user device 206 for access to the medical recommendations 216 by the user 212.

Referring now to FIG. 3, an operational flowchart illustrating the exemplary mobility assessment process 300 used by the mobility assessment program 110a, 110b according to at least one embodiment is depicted. According to one embodiment, FIG. 3 provides a general description of the mobility assessment process 300 with reference to the mobility assessment environment 200 in FIG. 2.

At 302, acceleration data associated with the user is received. According to one embodiment, the mobility assessment program 110a, 110b may be implemented to receive user movement data from one or more sensors. In one embodiment, the sensors may be implemented into devices such as, for example, a wearable smart device (e.g., a smart watch), adhesive patch, belt, and/or smart clothing. In one embodiment, the mobility assessment program 110a, 110b may only collect the movement data following user consent to having movement data collected by the mobility assessment program 110a, 110b. In one embodiment, the movement data may include acceleration data, herein defined as a rate of change in velocity over time, in any suitable rate format. In one embodiment, the sensors may include a triaxial accelerometer implemented to simultaneously measure user acceleration in one or more orthogonal directions. For example, the triaxial accelerometer may be implemented to simultaneously measure user acceleration in three orthogonal directions (e.g., x-axis; y-axis; and z-axis).

Then at 304, an activity state of the user is determined based on the received acceleration data. As described previously with reference to FIG. 2, the mobility assessment program 110a, 110b may determine the activity state using spectrum analysis approach and/or time-domain approach. If the mobility assessment program 110a, 110b implements the spectrum analysis approach, the mobility assessment program 110a, 110b may identify the peak frequency based on the spectral power density (e.g., function of frequency over a range of frequencies observed in the signal) and use the peak frequency to determine the respective activity state of the user.

If the mobility assessment program 110a, 110b implements the spectrum time-domain approach, the mobility assessment program 110a, 110b may calculate an activity intensity (AI) of the user based on the received acceleration data. According to one embodiment, the mobility assessment program 110a, 110b may calculate the AI using Equation 1, as described previously with reference to FIG. 2 and acceleration data measured in one or more orthogonal directions (e.g., measured using a triaxial accelerometer). In at least one embodiment, the mobility assessment program 110a, 110b may calculate the AI using acceleration data including simultaneous acceleration measurements in three orthogonal directions. In one embodiment, the mobility assessment program 110a, 110b may calculate the AI of the user at a regular time interval. More specifically, in one embodiment, the mobility assessment program 110a, 110b may calculate the AI using acceleration data collected (e.g., sampled) at regular time intervals. For example, the mobility assessment program 110a, 110b may calculate the AI using acceleration data collected every 15 seconds, using 30 second windows.

Then at 306, an amount of time spent in a respective activity state of a plurality of activity states is recorded. According to one embodiment, the mobility assessment program 110a, 110b may define a set of activity states (e.g., plurality of activity states), as described previously with reference to FIG. 2 and Table 2. In one embodiment, the set of activity states may include, for example, rest, sedentary, mild, moderate, impact, and/or jitter. In one embodiment, the activity states may be defined based on certain peak frequency thresholds, a described previously with reference to FIG. 2.

In at least one embodiment, the activity states may additionally, or alternatively, be defined based on certain AI thresholds observed during mobility assessment tests, e.g., six minute walk test (6MWT), for the user or across several cohorts (e.g., patients having same or similar disease conditions as the user) over multiple clinical visits. According to one embodiment, the mobility assessment program 110a, 110b may identify the activity state of the user based on comparing the current calculated AI of the user with the AI thresholds defining the set of activity states. Each corresponding bin for a respective activity state may include a minimum AI threshold and a maximum AI threshold. In one embodiment, the mobility assessment program 110a, 110b may identify the activity state by identifying the corresponding bin where the current calculated

AI of the user is greater than the minimum AI threshold and less than or equal to the maximum AI threshold.

According to one embodiment, the mobility assessment program 110a, 110b may calculate and record the amount or fraction of time spent by the user in a respective activity state of the set of activity states. In at least one embodiment, calculating the fraction of time in the respective activity states may depend on the specified time-window for which the effective mobility is being calculated. For example, if the effective mobility is calculated daily (e.g., specified time-window for effective mobility scoring is once per day; 24 hours), the fraction of time in each activity state may be calculated and recorded as the fraction (e.g., in minutes or hours) of a day. In another example, if the effective mobility is calculated hourly (e.g., specified time-window for effective mobility scoring is once per hour), the fraction of time in each activity state may be calculated and recorded as the fraction (e.g., in minutes) of an hour.

Then at 308, an effective mobility score of the user is determined for a specified time-window based on calculating a weighted sum of time spent in the plurality of activity states. According to one embodiment, the mobility assessment program 110a, 110b may calculate the effective mobility score for the user using Equation 2, as described previously with reference to FIG. 2. According to one embodiment, each of the activity states may include a corresponding weighting factor. In one embodiment, the mobility assessment program 110a, 110b may multiply the fraction of time spent in each of the activity states by the corresponding weighting factor and divide that aggregation by the sum of the weighting factors.

In one embodiment, the effective mobility score may be normalized for the specified time-window for which the effective mobility is being calculated. For example, if the effective mobility is calculated hourly, the mobility assessment program 110a, 110b may normalize the effective mobility at the hour level by dividing the effective mobility score by 60 minutes, as expressed by Equation 3 described with reference to FIG. 2. In another example, if the effective mobility is calculated once per day, the mobility assessment program 110a, 110b may normalize the effective mobility at the day level by dividing the effective mobility score by 60 minutes and then by 24 hours, as expressed by Equation 4 described with reference to FIG. 2.

According to one embodiment, the mobility assessment program 110a, 110b may automatically quantify a pain intensity experienced by the user in the specified time-window based on the determined effective mobility score of the user. In one embodiment, the quantified pain intensity may include a prediction based on the current effective mobility of the user. In one embodiment, the mobility assessment program 110a, 110b may quantify the pain intensity level by determining the functional relationship between pain intensity and effective mobility for the user, as described previously with reference to FIG. 2.

In one embodiment, the relationship between effective mobility and pain intensity for a given user may be expressed as a monotonic function where the pain intensity only increases with an increase in effective mobility or the pain intensity only decreases with an increase in effective mobility. In another embodiment, the relationship between effective mobility and pain intensity for a given user 212 may be expressed as a non-monotonic where the pain intensity decreases with an increase in effective mobility up to a certain threshold value of effective mobility and beyond that, the pain intensity increases with an increase in effective mobility or vice versa (e.g., pain intensity increases with an increase in effective mobility up to a certain threshold value of effective mobility and beyond that, the pain intensity decreases with an increase in effective mobility). According to one embodiment, the mobility assessment program 110a, 110b may determine whether the relationship between effective mobility and pain intensity for a given user may be expressed as a monotonic function or a non-monotonic function using machine learning techniques, as described previously with reference to FIG. 2.

According to one embodiment, the mobility assessment program 110a, 110b may generate one or more recommendations based on the predicted pain intensity level and/or the predicted disease severity experienced by the user based on the effective mobility score of the user. In one embodiment, the recommendations may include therapy recommendations based on latest/current pain intensity or disease severity level (e.g., determined based on effective mobility score) and inputs from SMEs, such as, the physician of the user. In at least one embodiment, the recommendations may include therapeutic interventions needed based on predicted pain intensity and/or disease severity level using current effective mobility data and learnt patient-specific functional relationship between effective mobility and pain intensity and/or disease severity level.

According to one embodiment, the mobility assessment program 110a, 110b may provide the user with the recommendations as well as a summary of the user's progress (e.g., dashboard 214) via the user device.

Accordingly, the mobility assessment program 110a, 110b may improve the functionality of a computer because the mobility assessment program 110a, 110b may enable the computer to passively and objectively measure a user's chronic pain intensity and/or disease severity using the user's movement data captured using a wearable sensor.

It may be appreciated that FIGS. 2 and 3 provide only an illustration of one embodiment and do not imply any limitations with regard to how different embodiments may be implemented. Many modifications to the depicted embodiment(s) may be made based on design and implementation requirements.

FIG. 4 is a block diagram 900 of internal and external components of computers depicted in FIG. 1 in accordance with an illustrative embodiment of the present invention. It should be appreciated that FIG. 4 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902a, b and external components 904 a, b illustrated in FIG. 4. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the mobility assessment program 110a in client computer 102, and the mobility assessment program 110b in network server 112, may be stored on one or more computer-readable tangible storage devices 916 for execution by one or more processors 906 via one or more RAMs 908 (which typically include cache memory). In the embodiment illustrated in FIG. 4, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the mobility assessment program 110a and 110b can be stored on one or more of the respective portable computer-readable tangible storage devices 920, read via the respective RAY drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The software program 108 and the mobility assessment program 110a in client computer 102 and the mobility assessment program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the mobility assessment program 110a in client computer 102 and the mobility assessment program 110b in network server computer 112 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

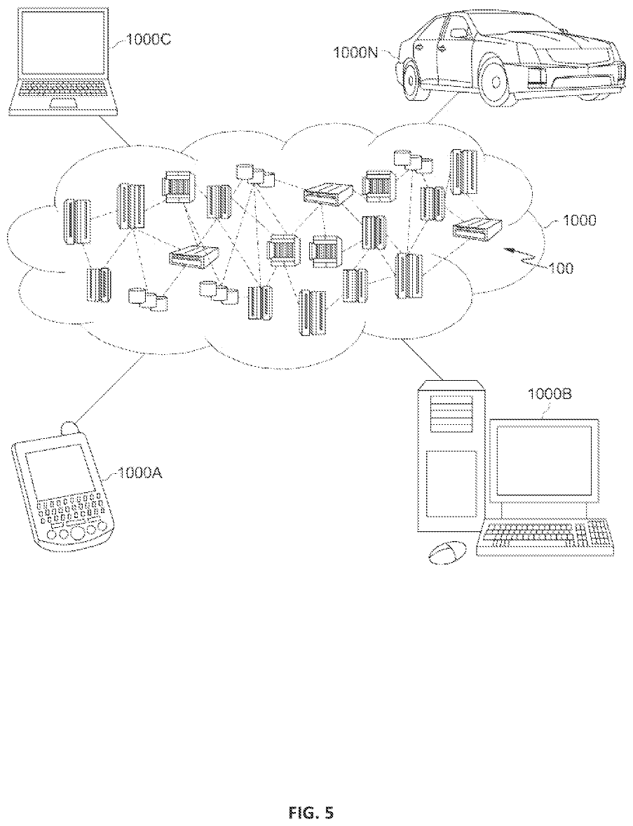

Referring now to FIG. 5, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 5 are intended to be illustrative only and that computing nodes 100 and cloud computing environment 1000 can communicate with any type of computerized device over any type of network and/or network addressable connection (e.g., using a web browser).

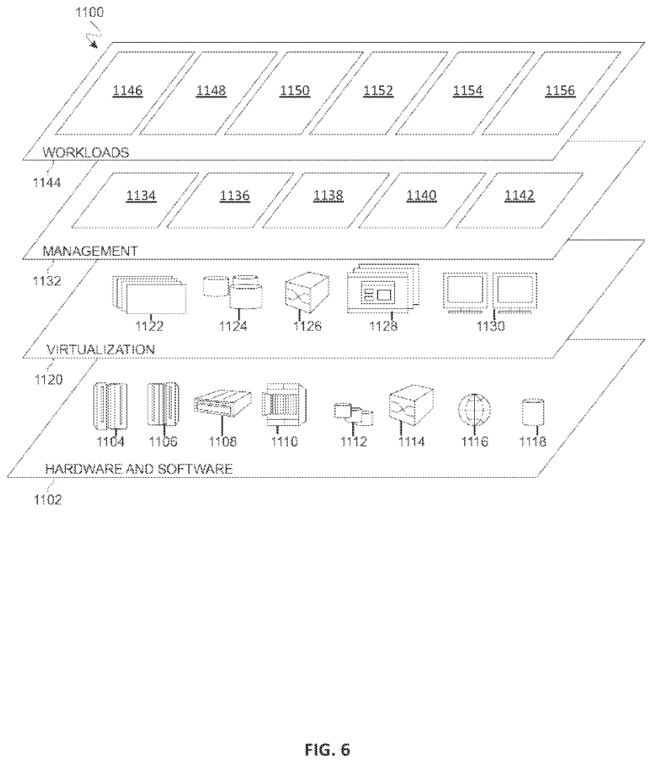

Referring now to FIG. 6, a set of functional abstraction layers 1100 provided by cloud computing environment 1000 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and mobility assessment 1156. A mobility assessment program 110a, 110b provides a way to determine an effective mobility score of a user for a specified time-window based on calculating a weighted sum of time spent in a plurality of activity states based on acceleration data.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
receiving an acceleration data associated with a user;
determining a respective activity state of a plurality of activity states for the user based on the received acceleration data;
recording an amount of time spent in the determined respective activity state of the plurality of activity states; and
determining an effective mobility score of the user for a specified time-window based on calculating a weighted sum of time spent in the plurality of activity states.

2. The method of claim 1, wherein determining the respective activity state of the plurality of activity states for the user based on the received acceleration data further comprises:
performing a spectrum analysis of the received acceleration data to produce a spectral power density;
identifying a peak frequency corresponding to a peak value of the spectral power density; and
determining the respective activity state based on the identified peak frequency.

3. The method of claim 1, wherein determining the respective activity state of the plurality of activity states for the user based on the received acceleration data further comprises:
calculating an activity intensity of the user based on the received acceleration data wherein the respective activity state is identified based on the calculated activity intensity.

4. The method of claim 1, further comprising:
automatically quantifying a pain intensity experienced by the user in the specified time-window based on the determined effective mobility score of the user.

5. The method of claim 1, further comprising:
measuring the received acceleration data using a triaxial accelerometer, wherein the received acceleration data includes a simultaneous measurement of user acceleration in three orthogonal directions.

6. The method of claim 3, wherein calculating the activity intensity of the user based on the received acceleration data further comprises:
calculating the activity intensity of the user at a regular time interval.

7. The method of claim 1, wherein receiving the acceleration data associated with the user further comprises:
receiving a time series of acceleration data sampled at a regular time interval.

8. The method of claim 3, further comprising:
defining each activity state of the plurality of activity states using a corresponding minimum activity intensity threshold and a corresponding maximum activity intensity threshold.

9. The method of claim 4, wherein automatically quantifying the pain intensity experienced by the user in the specified time-window based on the determined effective mobility score of the user further comprises:
determining a functional relationship between the determined effective mobility score of the user and the automatically quantified pain intensity experienced by the user.

10. The method of claim 9, wherein the determined functional relationship between the determined effective mobility score of the user and the automatically quantified pain intensity experienced by the user is selected from the group consisting of: a monotonic functional relationship and a non-monotonic functional relationship.

11. The method of claim 9, further comprising:
generating a medical recommendation for the user based on the automatically quantified pain intensity experienced by the user and the determined functional relationship between the determined effective mobility score of the user and the automatically quantified pain intensity experienced by the user.

12. A computer system for effective mobility scoring, comprising:
one or more processors, one or more computer-readable memories, one or more computer-readable tangible storage media, and program instructions stored on at least one of the one or more computer-readable tangible storage media for execution by at least one of the one or more processors via at least one of the one or more memories, wherein the computer system is capable of performing a method comprising:
receiving an acceleration data associated with a user;
determining a respective activity state of a plurality of activity states for the user based on the received acceleration data;
recording an amount of time spent in the determined respective activity state of the plurality of activity states; and
determining an effective mobility score of the user for a specified time-window based on calculating a weighted sum of time spent in the plurality of activity states.

13. The computer system of claim 12, wherein determining the respective activity state of the plurality of activity states for the user based on the received acceleration data further comprises:
performing a spectrum analysis of the received acceleration data to produce a spectral power density;
identifying a peak frequency corresponding to a peak value of the spectral power density; and
determining the respective activity state based on the identified peak frequency.

14. The computer system of claim 12, wherein determining the respective activity state of the plurality of activity states for the user based on the received acceleration data further comprises:
calculating an activity intensity of the user based on the received acceleration data wherein the respective activity state is identified based on the calculated activity intensity.

15. The computer system of claim 12, further comprising:
automatically quantifying a pain intensity experienced by the user in the specified time-window based on the determined effective mobility score of the user.

16. The computer system of claim 12, further comprising:
measuring the received acceleration data using a triaxial accelerometer, wherein the received acceleration data includes a simultaneous measurement of user acceleration in three orthogonal directions.

17. The computer system of claim 14, wherein calculating the activity intensity of the user based on the received acceleration data further comprises:
calculating the activity intensity of the user at a regular time interval.

18. The computer system of claim 12, wherein receiving the acceleration data associated with the user further comprises:
receiving a time series of acceleration data sampled at a regular time interval.

19. The computer system of claim 14, further comprising:
defining each activity state of the plurality of activity states using a corresponding minimum activity intensity threshold and a corresponding maximum activity intensity threshold.

20. A computer program product for effective mobility scoring, comprising:
one or more computer-readable storage media and program instructions collectively stored on the one or more computer-readable storage media, the program instructions executable by a processor to cause the processor to perform a method comprising:
receiving an acceleration data associated with a user;
determining a respective activity state of a plurality of activity states for the user based on the received acceleration data;
recording an amount of time spent in the determined respective activity state of the plurality of activity states; and
determining an effective mobility score of the user for a specified time-window based on calculating a weighted sum of time spent in the plurality of activity states.
